(19) United States
(12) Patent Application Publication
(10) Pub. No.: US 2017/0120059 A1
(43) Pub. Date: May 4, 2017

(54) MULTISITE PACING CAPTURE DETERMINATION BASED ON EVOKED RESPONSE

(71) Applicant: Cardiac Pacemakers, Inc., St. Paul, MN (US)

(72) Inventors: Sunipa Saha, Shoreview, MN (US); Keith L. Herrmann, Minneapolis, MN (US); Yinghong Yu, Shoreview, CA (US)

(21) Appl. No.: 15/335,237

(22) Filed: Oct. 26, 2016

Related U.S. Application Data
(60) Provisional application No. 62/248,683, filed on Oct. 30, 2015.

(51) Int. Cl. A61N 1/37; A61N 1/372; A61N 1/368; A61N 1/05

(52) U.S. Cl. CPC A61N 1/3712; A61N 1/056; A61N 1/3704; A61N 1/37252; A61N 1/3684

(57) ABSTRACT

An apparatus comprises a stimulus circuit, a cardiac signal sensing circuit, and a control circuit. The stimulus circuit provides electrical pulse energy to a first pacing channel that includes a first left ventricular (LV) electrode as a cathode and a second pacing channel that includes a second LV electrode as a cathode. The cardiac signal sensing circuit senses cardiac signals using a first sensing channel that includes one of the first LV electrode or the second LV electrode. The control circuit includes a capture detection sub-circuit configured to: initiate delivery of electrical pulse energy to both the first pacing channel and the second pacing channel; sense cardiac depolarization of a ventricle using the first sensing channel; determine first and second cardiac capture pulse energy level thresholds for the first and second pacing channels respectively; and provide indications of the cardiac capture pulse energy level thresholds to a user or process.

### CLAIM OF PRIORITY

This application claims the benefit of priority under 35 U.S.C. §119(e) of U.S. Provisional Patent Application Ser. No. 62/248,683, filed on Oct. 30, 2015, which is herein incorporated by reference in its entirety.

### BACKGROUND

Ambulatory medical devices can be used to treat patients or subjects using electrical or other therapy, or to aid a physician or caregiver in patient diagnosis through internal monitoring of a patient's condition. Some types of ambulatory medical devices may be implantable or partially implantable. Some examples include cardiac function management (CFM) devices such as implantable pacemakers, implantable cardioverter defibrillators (ICDs), cardiac resynchronization therapy devices (CRTs), and devices that include a combination of such capabilities. The devices may include one or more electrodes in communication with one or more sense amplifiers to monitor electrical heart activity within a patient, and often include one or more sensors to monitor one or more other internal patient parameters. The devices may be implanted subcutaneously and may include electrodes that are able to sense cardiac signals without being in direct contact with the patient's heart. Other examples of implantable medical devices (IMDs) include implantable diagnostic devices, implantable drug delivery systems, or implantable devices with neural stimulation capability (e.g., vagus nerve stimulator, baroreflex stimulator, carotid sinus stimulator, deep brain stimulator, sacral nerve stimulator, etc.).

Operation of an IMD is typically optimized for particular patient by a caregiver, such as by programming different device operating parameters or settings for example. Manufacturers of such devices continue to improve and add functionality to the devices, which can make them complicated to program. The inventors have recognized a need for improved optimization of device-based therapy.

### OVERVIEW

The present subject matter relates to providing multi-site electrical stimulation therapy. An example multi-site electrical stimulation therapy is multi-site pacing stimulation delivered to multiple sites within the same chamber of the heart.

An apparatus example of the present subject matter is for electrical coupling to a plurality of implantable electrodes. The apparatus example includes a stimulus circuit, a cardiac signal sensing circuit, and a control circuit. The stimulus circuit provides electrical pulse energy to at least a first pacing channel that includes a first left ventricular (LV) electrode as a cathode and a second pacing channel that includes a second LV electrode as a cathode. The cardiac signal sensing circuit senses cardiac activity signals using at least a first sensing channel that includes one of the first LV electrode or the second LV electrode. The control circuit includes a capture detection sub-circuit configured to: initiate delivery of electrical pulse energy to both the first pacing channel and the second pacing channel; sense cardiac depolarization of a ventricle using the first sensing channel; determine a first cardiac capture pulse energy level threshold for the first pacing channel; determine a second cardiac capture pulse energy level threshold for the second pacing channel; and provide indications of the first and second cardiac capture pulse energy level thresholds to a user or process.

Multi-site pacing of a heart chamber may add complexity to the programming of parameter values for electrical stimulation therapy. Device generated recommendations for values of parameters associated with the electrical stimulation may assist the clinician or physician to optimize the device for the needs of a specific patient.

This section is intended to provide a brief overview of subject matter of the present patent application. It is not intended to provide an exclusive or exhaustive explanation of the invention. The detailed description is included to provide further information about the present patent application such as a discussion of the dependent clams and the interrelation of the dependent and independent claims in addition to the statements made in this section.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, the various examples discussed in the present document.

FIG. 1 is an illustration of an example of portions of a system that includes an implantable medical device.

FIGS. 2A and 2B illustrate examples of sensing channels for an ambulatory medical device.

FIG. 3 shows an example of a method of controlling operation of an implantable medical device

FIG. 4 is a block diagram of portions of an example of a medical device to determine cardiac capture pulse energy level thresholds for multi-site pacing.

FIG. 5 illustrates portions of an example of a medical device system that includes electrodes and an implantable medical device configured for dual-site pacing.

FIG. 6 is a flow diagram of an example of a device-based method of determining pacing capture thresholds for multi-site pacing.

FIG. 7 illustrates portions of an example of a medical device system that includes a shared cathode sensing channel.

FIG. 8 is a graph used to illustrate the cardiac capture threshold determination of the method example of FIG. 6.

FIGS. 9A and 9B show an example of a shift in sensed cardiac signal morphology from shared cathode sensing to independent pacing and sensing.

FIG. 10 is a graph used to illustrate the cardiac capture threshold determination of the method example of FIG. 6.

FIG. 11 is a flow diagram of another example of a device-based method of determining pacing capture thresholds for multi-site pacing.

FIG. 12 is an illustration of portions of an example of a system that uses a deployed implantable medical device to provide a therapy to a patient.

### DETAILED DESCRIPTION

An ambulatory medical device can include one or more of the features, structures, methods, or combinations thereof described herein. For example, a cardiac function management device may be implemented to include one or more of the advantageous features or processes described below. It is intended that such a cardiac function management device, or other implantable or partially implantable device, need not include all of the features described herein, but may be implemented to include selected features that provide for unique structures or functionality. Such a device may be implemented to provide a variety of therapeutic or diagnostic functions.

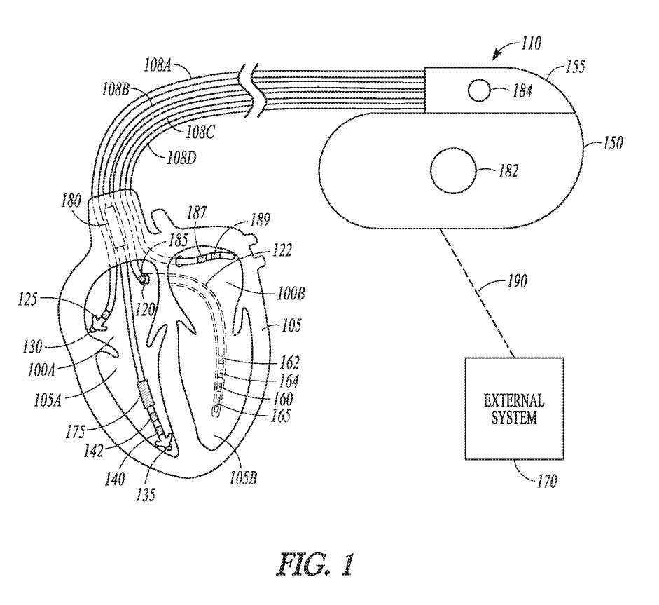

FIG. 1 is an illustration of portions of a system that includes an ambulatory medical device that is an IMD 110. Examples of IMD 110 include, without limitation, a pacemaker, a defibrillator, a cardiac resynchronization therapy (CRT) device, or a combination of such devices. In other examples, the IMD is a neurostimulator such as among other things a vagus nerve stimulator, baroreflex stimulator, carotid sinus stimulator, deep brain stimulator, or sacral nerve stimulator. The system 100 also typically includes an IMD programmer or other external system 170 that communicates wireless signals 190 with the IMD 110, such as by using radio frequency (RE) or other telemetry signals.

The MID 110 can be coupled by one or more leads 108A-D to heart 105. Cardiac leads 108A-D include a proximal end that is coupled to IMD 110 and a distal end, coupled by electrical contacts or “electrodes” to one or more portions of a heart 105. The electrodes typically deliver cardioversion, defibrillation, pacing, or resynchronization therapy, or combinations thereof to at least one chamber of the heart 105. The electrodes may be electrically coupled to sense amplifiers to sense electrical cardiac signals. Sometimes the sensing circuits and electrodes are referred to as sensing channels. For example, circuitry used to sense signals in an atrium is referred to as an atrial sensing channel, and circuitry used to sense signals in a ventricle is referred to as a ventricular sensing channel. When direction is taken into account due to position of one or more sensing electrodes, the sensing channel can be referred to as a sensing vector.

Sensed electrical cardiac signals can be sampled to create an electrogram. An electrogram can be analyzed by the IMD and/or can be stored in the IMD and later communicated to an external device where the sampled cardiac signals can be displayed for analysis.

Heart 105 includes a right atrium 100A, a left atrium 100B, a right ventricle 105A, a left ventricle 105B, and a coronary sinus 120 extending from right atrium 100A. Right atrial (RA) lead 108A includes electrodes (electrical contacts, such as ring electrode 125 and tip electrode 130) disposed in an atrium 100A of heart 105 for sensing signals, or delivering pacing therapy, or both, to the atrium 100A. Electrodes used to provide pacing therapy can be referred to as pacing channels. When direction is taken into account due to position of one or more pacing electrodes, the pacing channel can be referred to as a pacing vector. Similarly, electrodes used to sense cardiac signals with sense amplifiers can be referred to as sensing channels, and when direction is taken into account a sensing channel can be referred to as a sensing vector.

Right ventricular (RV) lead 108B includes one or more electrodes, such as tip electrode 135 and ring electrode 140, for sensing signals, delivering pacing therapy, or both sensing signals and delivering pacing therapy. RV lead 108B can include one or more additional ring electrodes 142 to provide multi-site pacing to the RV. Lead 108B optionally also includes additional electrodes, such as electrodes 175 and 180, for delivering atrial cardioversion, atrial defibrillation, ventricular cardioversion, ventricular defibrillation, or combinations thereof to heart 105. Such electrodes typically have larger surface areas than pacing electrodes in order to handle the larger energies involved in defibrillation. Lead 108B optionally provides resynchronization therapy to the heart 105. Resynchronization therapy is typically delivered to the ventricles in order to better synchronize the timing of depolarizations between ventricles.

The IMD 110 can include a third cardiac lead 108C attached to the IMD 110 through the header 155. The third cardiac lead 108C includes electrodes 160, 162, 164, and 165 placed in a coronary vein 122 lying epicardially on the left ventricle (LV) 105B via the coronary vein. The number of electrodes shown in the Figure is only an example and other arrangements are possible. For instance, the third cardiac lead 108C may include less electrodes (e.g., one or two electrodes) or more electrodes (e.g., eight or more electrodes) than the example shown, and may include a ring electrode 185 positioned near the coronary sinus (CS) 120. LV lead 108C can provide multi-site pacing to the LV.

In addition to cardiac leads 108A, 108B, 108C, or in the alternative to one or more of cardiac leads 108A, 108B, 108C, the IMD 110 can include a fourth cardiac lead 108D that includes electrodes 187 and 189 placed in a vessel lying epicardially on the left atrium (LA) 100B.

The IMD 110 can include a hermetically-sealed IMD housing or can 150, and the MID 110 can include an electrode 182 formed on the IMD can 150. The IMD 100 may include an IMD header 155 for coupling to the cardiac leads, and the IMD header 155 may also include an electrode 184. Cardiac pacing therapy can be delivered in a unipolar mode using the electrode 182 or electrode 184 and one or more electrodes formed on a lead. Cardiac pacing therapy can be delivered in an extended bipolar pacing mode using only one electrode of a lead (e.g., only one electrode of LV lead 108C) and one electrode of a different lead (e.g., only one electrode of RV lead 108B). Cardiac pacing therapy can be delivered in a monopolar pacing mode using only one electrode of a lead without a second electrode.

Lead 108B can include a defibrillation RV coil electrode 175 located proximal to tip and ring electrodes 135, 140, and a second defibrillation coil electrode 180 located proximal to the RV coil electrode 175, tip electrode 135, and ring electrode 140 for placement in the superior vena cava (SVC). In some examples, high-energy shock therapy is delivered from the first or RV coil 175 to the second or SVC coil 180. In some examples, the SVC coil 180 is electrically tied to the electrode 182 formed on the IMD can 150. This improves defibrillation by delivering current from the RV coil electrode 175 more uniformly over the ventricular myocardium. In some examples, the therapy is delivered from the RV coil 175 only to the electrode 182 formed on the IMD can 150. In some examples, the coil electrodes 175, 180 are used in combination with other electrodes for sensing signals.

Note that the specific arrangement of leads and electrodes shown in the illustrated example of FIG. 1 is intended to be non-limiting. An IMD can be configured with a variety of electrode arrangements including transvenous, endocardial, and epicardial electrodes (e.g., an epicardial patch that may include dozens of electrodes , and/or subcutaneous, non-intrathoracic electrodes. An IMD 110 can be connectable to subcutaneous array or lead electrodes (e.g., non-intrathoracic electrodes or additional LV leads implantable along the LV wall, and leads implantable in one or both atria) that can be implanted in other areas of the body to help “steer” electrical currents produced by IMD 110. An IMD can be leadless (e.g., a leadless pacemaker). A leadless IMD may be placed in a heart chamber (e.g., RV or LV) and multiple electrodes of the leadless IMD may contact multiple tissue sites of the heart chamber.

Electrical pacing therapy is provided by a CRM device in response to an abnormally slow heart rate to induce cardiac depolarization and contraction (sometimes called capture of the heart, or cardiac capture). Pacing stimulation energy is delivered to provide a depolarization rate that improves hemodynamic function of the patient. The pacing stimulation energy should be optimized to produce cardiac capture. If the pacing stimulation energy is too high, stimulation to multiple pacing sites may cause stress on the heart and the battery life of an implanted device will be needlessly short. Also, pacing stimulation energy that is too high may cause unwanted non-cardiac stimulation such as stimulation of the phrenic nerve or stimulation of muscle near the tissue pocket in which the device is implanted. If the pacing stimulation energy is too low, the pacing energy will not evoke a response in the heart and the device will not produce the desired heart contractions.

To determine the appropriate electrostimulation energy, a CRM device may deliver a sequence of electrostimulation pulses to the heart as part of an automatic cardiac capture test. The sequence may include a successive reduction of the energy of the electrostimulation pulses. A first electrostimulation pulse that will induce cardiac capture is delivered. The level of energy of subsequent electrostimulation pulses is reduced in steps until the device verifies that failure to induce capture has occurred. Alternatively, the sequence may include increasing the energy level of the electrostimulation pulses. A first electrostimulation pulse that is below a threshold likely to induce capture is delivered. The energy of subsequent electrostimulation pulses is increased in steps until the device verifies that capture was induced. In another approach, the CRM device may increase and decrease the sequence of stimulation pulses as part of the cardiac capture test. As explained previously herein, the CRM device may sense cardiac signals using one or more of the sensing channels. To verify cardiac capture, the device may be able to distinguish an electrical signal that shows an evoked response from an electrical that shows no evoked response. Correctly identifying evoked response in the cardiac tissue target can be used detect a change from no capture to capture, or a change from capture to loss of capture.

When the minimum energy required to cause capture of the tissue target is determined, a safety margin can be added to the minimum energy to ensure efficacy of the stimulation pulses. An approach to determining a safety margin for device delivery of electrical pacing therapy can be found in Brisben et al., U.S. Pat. No. 8,565,879, “Method and Apparatus for Pacing Safety Margin,” filed Mar. 25, 2011, which is incorporated herein by reference in its entirety.

As explained previously herein, a CRM device may be configured to provide multi-site pacing, in which electrical stimulation pulses are provided to multiple sites within the same heart chamber. For example, in the electrode configuration shown in FIG. 1, pacing can be provided to the LV using a first pacing channel that includes can electrode 182 as the pacing channel anode and LV electrode 165 as the pacing channel cathode, and using a second pacing channel that includes the can electrode 182. and LV electrode 164. This may be useful to improve coordination of a contraction of the heart chamber, especially contraction of the left ventricle.

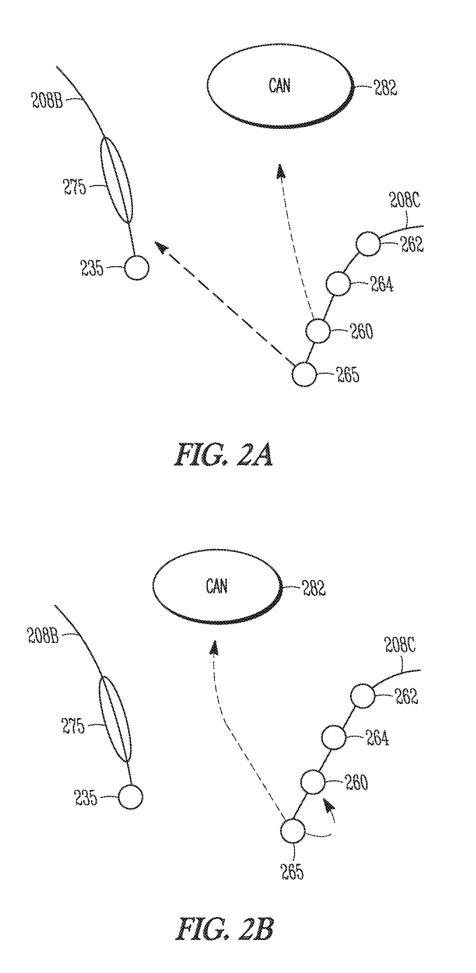

FIGS. 2A and 2B illustrate examples of sensing channels for an ambulatory medical device. Sensing channels can be configured (e.g., by position in the heart chamber) to sense cardiac activity used to detect whether multi-site pacing stimulation energy evoked a response in the cardiac tissue. A sensing channel may include electrodes separate from the electrodes used to deliver the pacing stimulation energy to provide independent pacing and sensing channels. FIG. 2A is an illustration of an example of electrodes configured in independent pacing and sensing channels. The pacing channel includes can electrode 282 as the pacing anode and electrode 260 of the LV lead 208C as the pacing cathode. The sensing channel includes RV coil electrode 275 of the RV lead 208A as the sensing anode and LV electrode 265 as the sensing cathode. One or more of LV electrodes 260, 264, and 262 may be included in additional pacing or sensing channels,

In some variations, the sense channel may include one or more electrodes used to deliver the pacing stimulation energy in a shared cathode sensing configuration. FIG. 2B is an illustration of an example of electrodes configured in a shared cathode sensing configuration. The pacing channel can include LV electrode 260 as the pacing anode and LV electrode 265 as the pacing cathode. The sensing channel can include can electrode 282 as the sensing anode and electrode 265 as the sensing cathode.

Returning to FIG. 1, the example shows that there can be several pacing channels available for multi-site pacing. A clinician may want information on multiple sites to appropriately customize the performance of a device to the specific patient's needs. Medical device based tests can be performed to automatically determine the appropriate electrostimulation energy for pacing therapy delivered using the multiple tissue sites. This can simplify the process of optimizing the device for a specific patient.

However, multi-site pacing can complicate device-based detection of evoked response as part of a cardiac capture test. The mode of the paced contraction may change as stimulation pulse energy is changed so that a response is evoked at both cardiac tissue sites of the heart chamber, at only one cardiac tissue site, or at no cardiac tissue sites. The morphology of sensed cardiac signals can be used to determine which tissue sites induced cardiac capture for the applied electrical stimulus.

Device-based morphology analysis can detect subtle changes in morphology. For instance, the medical device may determine a correlation value between electrogram signals sensed between successive steps of a capture threshold test. An example of a correlation value is feature correlation coefficient (FCC). The FCC can provide an indication of a degree of similarity between the shapes of the electrogram signals. When the FCC value changes to a value greater than a specified FCC threshold value, the morphology between the successive electrogram signals may have changed sufficiently to indicate a shift from multi-site capture to single-site capture. An approach to calculating a correlation value can be found in Kim et al., U.S. Pat. No. 7,904,142 filed May 16, 2007, which is incorporated herein by reference in its entirety. Device-based detection can be easier than visual or manual detection by a clinician and device-based detection can be performed in real time as the stimulation threshold test is performed.

An individual capture test could be performed separately on each electrode separately. However, a cardiac capture test may obtain better results if the test is performed while pacing the heart chamber with multiple electrodes if multi-site pacing is intended. For instance, using multi-site pacing during the capture test may show whether the delay between the multiple sites (if any) is appropriate. For the case of two pacing sites (or dual-site pacing), if the inter-site delay is appropriate, the pacing energy will induce cardiac capture at both pacing sites. If the inter-site delay is not appropriate, the pacing energy will not induce cardiac capture at both pacing sites because, for example, the later tissue site may be in refractory due to the pacing at the first tissue site.

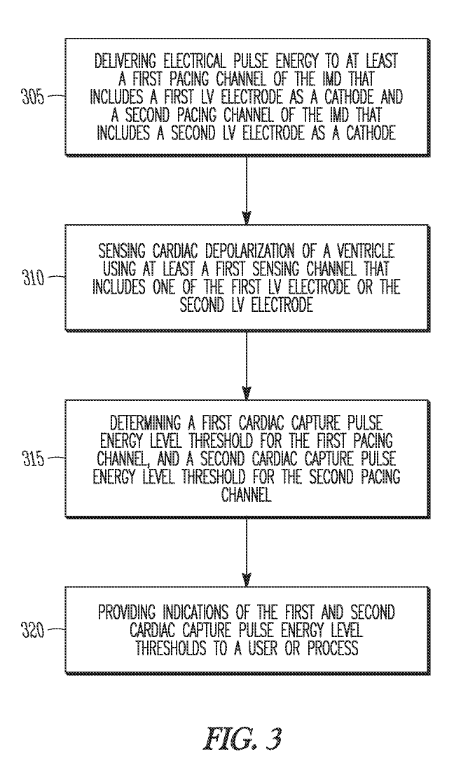

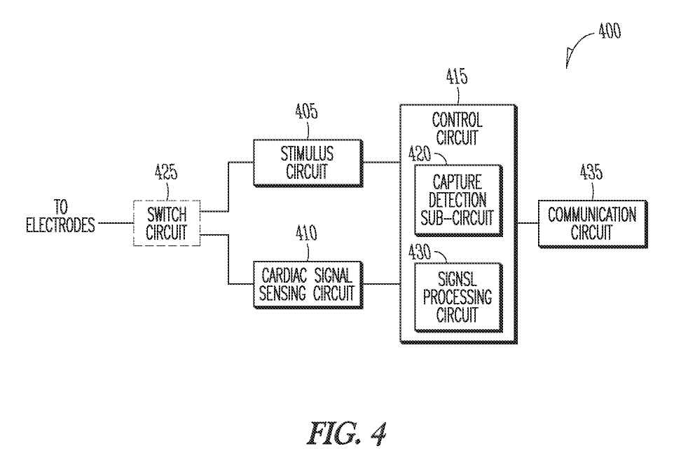

FIG. 3 is a flow diagram of an example of a method 300 of controlling operation of an IMD. At 305, electrical pulse energy is delivered to at least a first pacing channel of the IMD and a second pacing channel of the IMD The first pacing channel includes a first LV electrode as the cathode of the first pacing channel, and the second pacing channel includes a second LV electrode as the cathode of the second pacing channel. In an illustrative example intended to be non-limiting, the first pacing channel may include the can electrode 182 formed on the implantable housing of FIG. 1 as the pacing anode and LV electrode 165 as the pacing cathode. The second pacing channel may include the can electrode 182 as the pacing anode and LV electrode 160 as the pacing cathode. At 310, cardiac depolarization of the ventricle is sensed using at least a first sensing channel that includes one of the first LV electrode or the second LV electrode. For instance, the sensing channel may include RV coil electrode 175 of FIG. 1 as the sensing anode and LV electrode 160 as the sensing cathode.

At 315, a first cardiac capture pulse energy level threshold is determined for the first pacing channel, and a second cardiac capture pulse energy level threshold for the second pacing channel. The cardiac capture pulse energy level threshold may be determined using a capture test performed by the IMD. At 320, indications of the first and second cardiac capture pulse energy level thresholds are provided to a user or process. An indication of an energy level threshold may include a signal or a value provided to a process executing on the same medical device or a separate medical device. In some variations, the indications are stored in a memory of the IMD and later uploaded to a separate memory device. In some variations, the one or more indications of pacing energy level are provided to a separate medical device for display to a user.

FIG. 4 is a block diagram of portions of an example of a medical device to determine cardiac capture pulse energy level thresholds for multi-site pacing. The device 400 includes a stimulus circuit 405 and a cardiac signal sensing circuit 410 that can be electrically coupled to implantable electrodes. The stimulus circuit 405 provides electrical pulse energy to at least a first pacing channel that includes a first LV electrode as a cathode and a second pacing channel that includes a second LV electrode as a cathode. The cardiac signal sensing circuit 410 senses cardiac activity signals using at least a first sensing channel that includes one of the first LV electrode or the second LV electrode. The device 400 may also include switch circuit 425 to electrically couple different combinations of the electrodes to the stimulus circuit 405 and the cardiac signal sensing circuit 410.

The device 400 also includes a control circuit 415 electrically coupled to the cardiac signal sensing circuit 410 and the stimulus circuit 405. The control circuit 415 can include can include a microprocessor, a digital signal processor, application specific integrated circuit (ASIC), or other type of processor, interpreting or executing instructions included in software or firmware. The control circuit 415 can include sub-circuits to perform the functions described. These sub-circuits may include software, hardware, firmware or any combination thereof. Multiple functions can be performed in one or more of the sub-circuits as desired.

The control circuit 415 includes a capture detection sub-circuit 420 that performs a capture test to determine the cardiac capture pulse energy level thresholds. The capture detection sub-circuit 420 initiates delivery of electrical pulse energy to both the first pacing channel and the second pacing channel and senses cardiac depolarization of a ventricle using the first sensing channel.

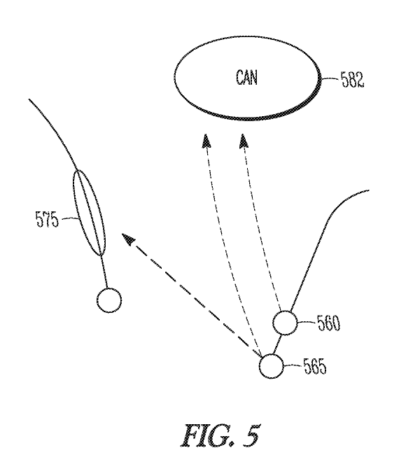

FIG. 5 illustrates portions of an example of a medical device system 500 that includes electrodes and an implantable medical device configured for dual-site pacing. The example shows a first pacing channel that includes LV electrode 565 and can electrode 582, and a second pacing channel that includes LV electrode 560 and can electrode 582. The example also shows a sensing channel that includes LV electrode 565 and RV coil electrode 575. Electrode 565 may be a cathode shared by the first pacing channel and the sensing channel, and the configuration uses one sense channel to sense the evoked response from the electrical pulse energy.

Returning to FIG. 4, the capture detection sub-circuit 420 determines a first cardiac capture pulse energy level threshold for the first pacing channel, and a second cardiac capture pulse energy level threshold for the second pacing channel. The capture detection sub-circuit 420 then provides the first and second cardiac capture pulse energy level thresholds to a user or process.

The electrical pulse energy may be delivered as part of a cardiac capture test performed by the IMD to determine the appropriate pulse energy level thresholds. During the test, the capture detection sub-circuit 420 initiates a change in pulse energy level of the delivered electrical pulse energy. The pacing energy level may be stepped up, stepped down, or step both up and down by the capture detection sub-circuit 420 during the test. One or more cardiac activity signals are sensed in association with a change from a first level of electrical pulse energy to a second level of electrical pulse energy. The sensed cardiac activity signals are then analyzed to determine if there is a change in the evoked response due to the change in pulse energy, such as a change from capture to loss of capture, or from no capture to capture.

A change in the evoked response can be detected by a change in morphology of the signal. When sensing cardiac activity signals using a channel having a cathode shared with a pacing channel, the morphology of the sensed signals exhibits a distinct change when the response changes between no capture or loss of capture), single-site capture, and dual-site capture. In some examples, the control circuit 415 includes a signal processing sub-circuit 430 that identifies a change in morphology in the sensed cardiac activity signals.

Using the identified change in morphology, the capture detection sub-circuit 420 distinguishes cardiac capture by one of the first and second pacing channels from cardiac capture by both pacing channels. For instance, the dominant peak of the signal may change one or more of polarity, magnitude, and width. The capture detection sub-circuit 420 may also distinguish cardiac capture by one or both of the first and second pacing channels from loss of capture by both pacing channels. For instance the timing of the dominant peak may change when the pacing energy fails to induce an evoked response and the peak in the sensed cardiac activity signal is an intrinsic response occurring later in the cardiac activity signal.

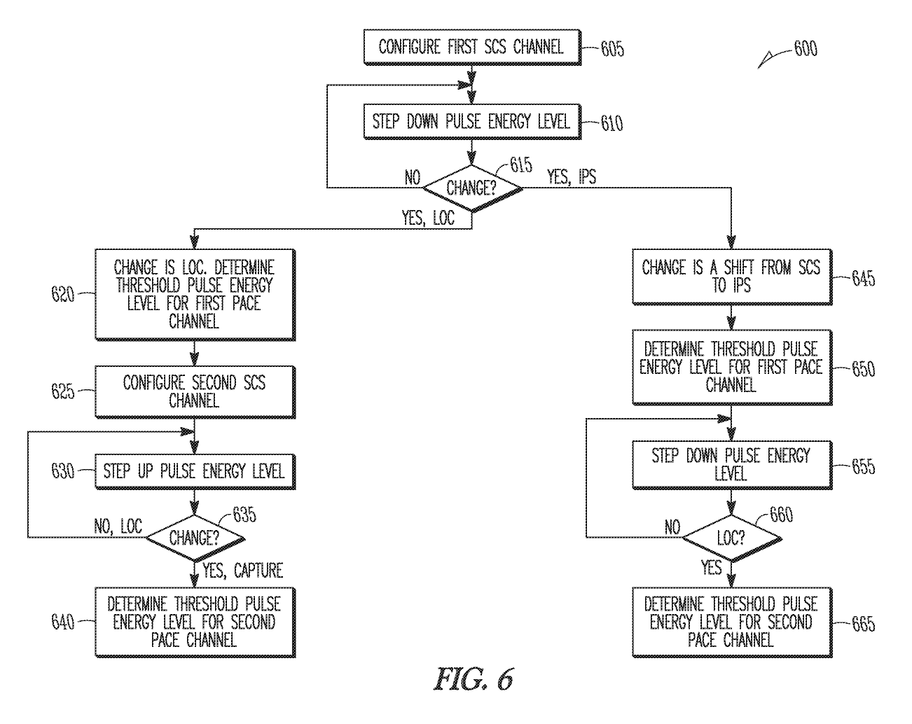

FIG. 6 is a flow diagram of an example of a device-based method 600 of determining pacing capture thresholds for dual-site pacing. The dual-site pacing may include two pacing channels such as the pacing channels shown in the example of FIG. 5, where the first pacing channel includes LV electrode 565 as the cathode and can electrode 582 as the anode, and the second pacing channel includes LV electrode 560 as the cathode and can electrode 582 as the anode.

At 605, an evoked response sensing channel is configured as a shared cathode sensing (SCS) channel. The SCS channel may be configured as shown in FIG. 5 with LV electrode 565 as the shared cathode and either the can electrode 582 or an electrode located in the RV (e.g., RV coil electrode 575) as the sensing channel anode. When the pacing channels and the sensing channel are configured (e.g., by the control circuit 415 of FIG. 4), a capture test is initiated and pulse energy is delivered to the two pacing channels and cardiac activity signals are sensed only with the one shared cathode sensing channel.

At 610, the pulse energy level is changed. In some examples, the pulse energy starts with a high energy level so that both pacing channels induce an evoked response at the cardiac tissue sites, and the pulse energy level is then stepped down at both cardiac tissue sites corresponding to LV electrode 565 and LV electrode 560. At each change in the pulse energy level, one or more cardiac activity signals are sensed with the SCS channel. The device may perform the same change in pulse level more than once during the capture test.

At 615, the stepping of the pulse energy level continues until there is a detected change in signal morphology. The device may look for a change in morphology in a specified time relation to the change in pulse energy level to ensure causality by the pulse energy level change.

Because the test began with capture by both pacing channels, the stepping down of the pulse energy level will eventually result in loss of capture at the first pacing channel or the second pacing channel. If cardiac capture is lost at the second pacing channel first, the SCS channel will still detect capture because the cathode is shared with the first pacing channel. Further stepping down of the pulse energy level will result in loss of capture by both pacing channels and the sensed signal morphology will show total loss of capture. If cardiac capture is first lost at the first pacing channel, the sensed signal morphology will appear to be sensing associated with independent pacing and sensing (IPS) channels because capture continues at the second pacing channel which does not share a cathode with the sensing channel. When a change in the sensed signals is detected, the capture detection sub-circuit 420 identifies the detected change in signal morphology.

If the change in morphology corresponds to a shift from a morphology indicating cardiac capture sensed by a shared cathode sensing channel to a morphology indicating loss of capture (LOC) by the pacing channel, the method 600 follows the left branch of the flow diagram to 620. The capture detection sub-circuit 420 may identify the change as a change to LOC morphology by a shift in time of the dominant peak of the sensed cardiac activity signal. The shift in time may occur because the new dominant peak is associated with an intrinsic response that occurs later than the pace pulse.

At 620, the threshold pulse energy level is determined for the first pacing channel. For instance, the pulse energy level just prior to that which resulted in loss of capture is recorded. A safety margin may then be added to the recorded pulse energy level to set the threshold pulse energy level.

At 625, the cardiac signal sensing circuit 410 is configured to sense cardiac activity signals using only a second sensing channel. The second sensing may also be a SCS channel that shares a cathode with the second pacing channel.

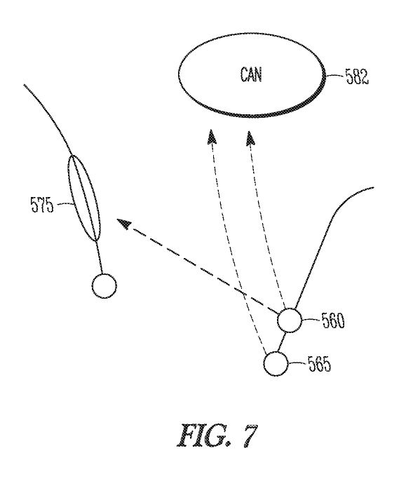

FIG. 7 shows the example of FIG. 5 with a second shared cathode sensing channel comprising RV coil electrode 575 as the sensing node and LV electrode 560 as the shared cathode. In certain examples, the first shared cathode sensing channel is disabled and sensing continues with the second shared cathode sensing channel. in certain examples, the first shared cathode sensing channel is reconfigured by the control circuit 415 to share the cathode with the second pacing channel.

Returning to FIG. 6, subsequent cardiac activity signals are then sensed using the second shared cathode sensing channel. The change in the signal morphology may have occurred because capture was first lost by the second pacing first, or both the first and second pacing channels lost capture by the same change in pulse energy.

At 630, the pulse energy level is stepped up to find the threshold for the second pacing channel. At 635, capture is detected when the morphology of the sensed cardiac activity signals changes from LOC to capture sensed with SCS by the second sensing channel. The pulse energy level that resulted in capture by the second pacing channel is recorded at 640. The pulse energy level threshold for the second pacing channel may be determined by adding a safety margin to the recorded pulse energy level.

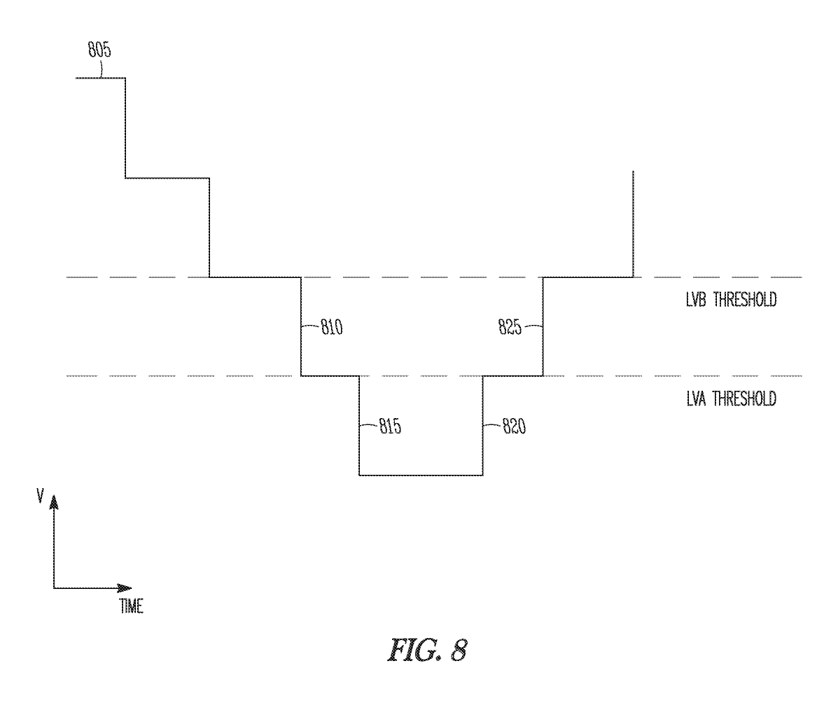

FIG. 8 is a graph that illustrates the threshold determination in the left branch of the method 600 of FIG. 6. In the graph, the vertical direction represents pulse energy and the horizontal direction represents time. At the top left of the graph, evoked response (ER) in the cardiac activity signals is sensed using the first shared cathode sensing channel. At 805, cardiac capture by both pacing channels is evident in the sensed cardiac activity signals and has a morphology corresponding to cardiac capture sensed by a shared cathode sensing channel. Moving to the right in the graph, the pulse energy level is stepped down.

At 810, cardiac capture is lost by the second pacing channel (e.g., LV electrode 560 in FIG. 5). The pulse energy level continues to be stepped down. At 815, cardiac capture is lost by the first pacing channel (e.g., LV electrode 565 in FIG. 5) and neither pacing channel captures the heart. The LOC may be detected as a shift to a signal morphology that is associated with complete loss of capture. The pulse energy level just prior to loss of capture by the first pacing channel is recorded. The sensing of the cardiac activity signals is changed over to the second shared cathode sensing channel. There is no evidence of capture by either pacing channel in cardiac activity signals sensed with the second shared cathode sensing channel at this point. The pulse energy level is stepped up.

At 820, capture by the first pacing channel may be evident in cardiac activity signals sensed with the second shared cathode sensing channel. The morphology would be associated with IPS because the second sensing channel does not share a cathode with the first pacing channel. At 825, cardiac capture by both the first and second pacing channels is evident in cardiac activity signals sensed with the second shared cathode sensing channel, The pulse energy level that induced cardiac capture by the second pacing channel is recorded.

Returning to FIG. 6, as explained previously, a change in morphology is detected at 615 and the capture detection sub-circuit 420 identifies the change. At this point in the flow diagram, the shared cathode sensing channel still shares the cathode with the first pacing channel (e.g., LV electrode 565 in FIG. 5). If the change in morphology corresponds to a change from a morphology indicating capture sensed using a SCS channel to a morphology indicating capture sensed using an TIPS channel, the method 600 branches to the right at 645 where the capture detection sub-circuit 420 identifies the change as SCS to IPS.

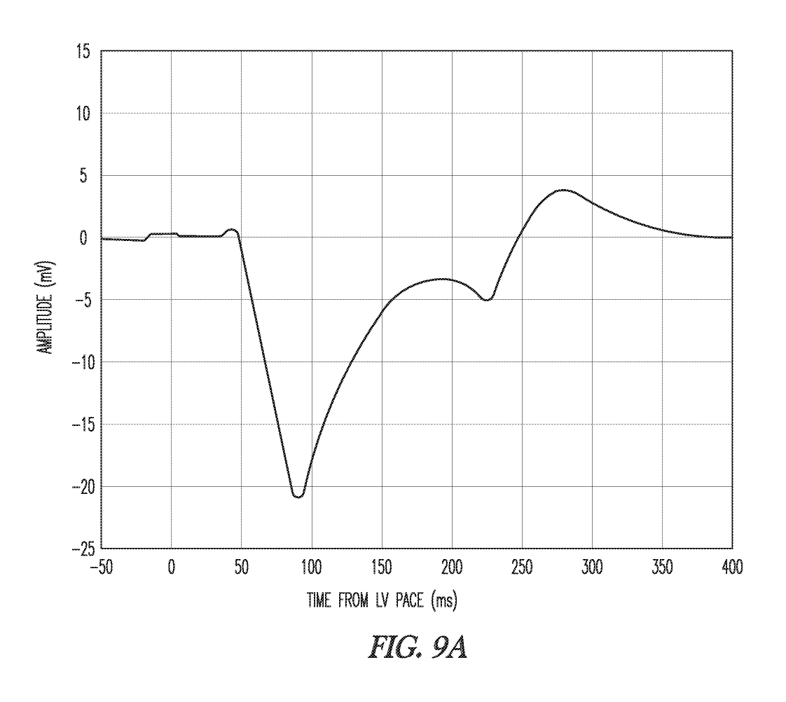

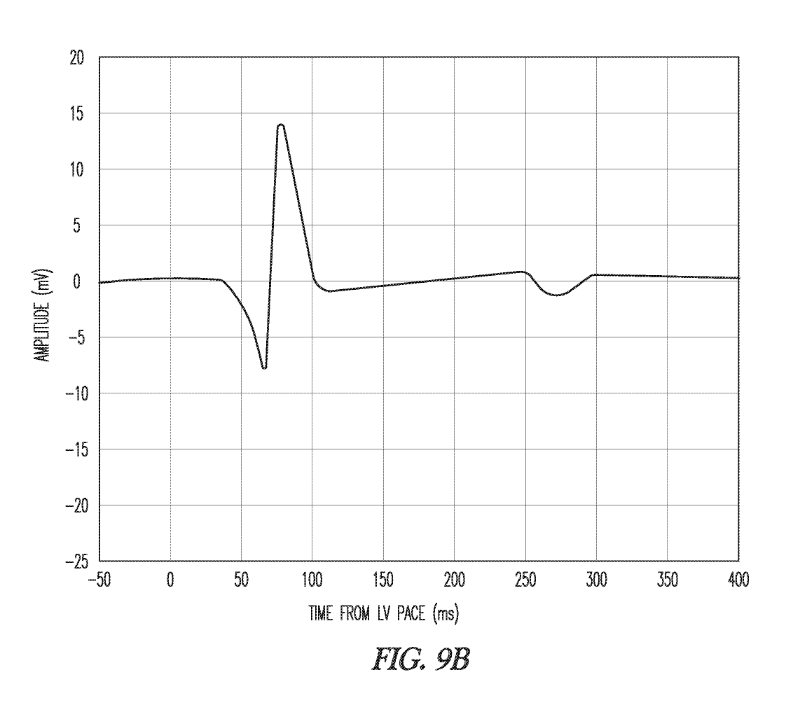

FIGS. 9A and 9B show an example of a shift in sensed cardiac signal morphology from SCS to IPS. FIG. 9A shows an example of morphology of capture sensed with SCS. The example shows one dominant peak in the morphology and in the example the peak is negative in amplitude. FIG. 9B shows an example of morphology of capture sensed with IPS. The example shows a bimodal morphology with a dominant positive peak. Thus, in the examples of

FIGS. 9A and 9B, the capture detection sub-circuit 420 identifies the change from SCS to IPS based on the detected shift in morphology from FIG. 9A to FIG. 9B.

Returning to FIG. 6 at 650, the pulse energy level threshold just prior to that which resulted in the shift from SCS morphology to IPS morphology is recorded (e.g., stored in device memory) for the first pacing channel, A safety margin may be added to the recorded energy level to set the pulse energy threshold for the first pacing channel. At 655, the step down of the pulse energy level continues until the sensed cardiac activity signals indicate LOC. The capture detection sub-circuit 420 may identify the change from IPS to LOC at 660 from the shift in signal morphology. The pulse energy level just prior to that which resulted in the shift to LOC is recorded for the second pacing channel. Note that the shared cathode sensing channel in the right branch of the method of FIG. 6 is not changed as it was for the left branch of FIG. 6. At 665, the pulse energy level is determined using the recorded pulse energy level.

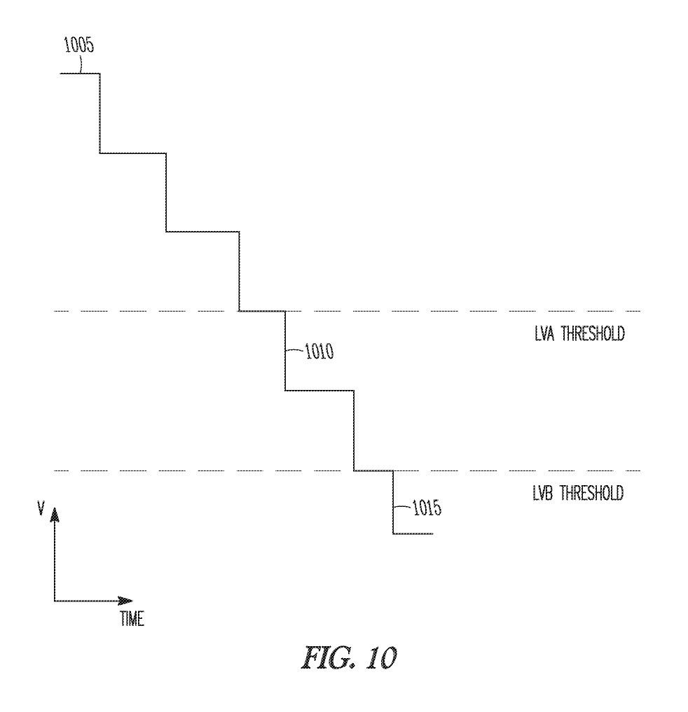

FIG. 10 is a graph that illustrates the threshold determination in the right branch of the method of FIG. 6. As in the graph of FIG. 8, the vertical direction represents pulse energy and the horizontal direction represents time. At the top left of the graph, the cardiac activity signals are sensed using the first shared cathode sensing channel (e.g., LV electrode 565 in FIG. 5 is shared by the first sensing channel and the first pacing channel). At 1005, cardiac capture by both pacing channels is evident in the sensed cardiac activity signals and has a signal morphology indicating cardiac capture sensed by a SCS channel. Moving to the right in the graph, the pulse energy level is stepped down.

At 1010, cardiac capture is lost by the first pacing channel and the morphology of sensed cardiac signal changes from a morphology indicating capture sensed using SCS to a morphology indicating capture sensed using IPS. The second pacing channel (e.g., a pacing channel with LV electrode 560 in FIG. 5) continues to capture the heart. The pulse energy level delivered just prior to the pulse energy level that resulted in the morphology shift is recorded and is used to determine the pulse energy threshold for the first pacing channel.

At 1015, cardiac capture is lost by the second pacing channel, and the morphology of sensed cardiac activity signals change from a morphology indicating capture sensed using IPS to a morphology indicating LOC. The pulse energy level delivered to the pulse energy level that resulted in LOC is recorded and is used to determine the pulse energy threshold for the second pacing channel.

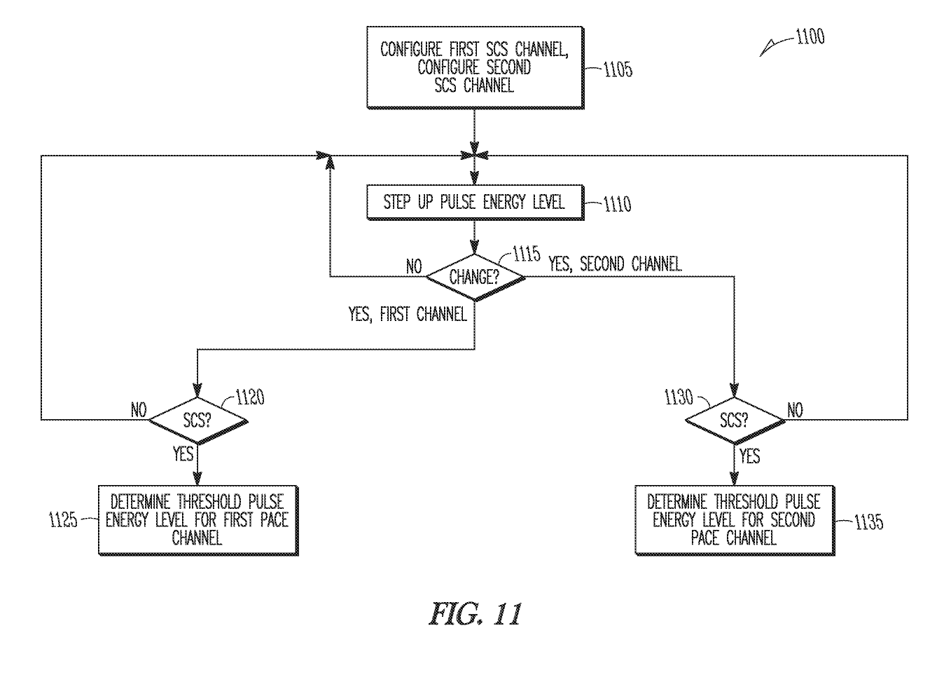

FIG. 11 is a flow diagram of another example of a method 1100 of determining pacing capture thresholds for dual-site pacing. The dual-site pacing may include two pacing channels and two sensing channels. For dual-site pacing in the LV, the stimulus circuit 405 of FIG. 4 provides pulse energy to a first pacing channel that includes a first LV electrode (e.g., LV electrode 565 in FIG. 5) and the cardiac signal sensing circuit 410 senses cardiac activity signals using a first sensing channel that includes the first LV electrode as a sensing cathode. The stimulus circuit 405 also provides pulse energy to a second pacing channel that includes a second LV electrode (e.g., LV electrode 560 in FIG. 5) and the cardiac signal sensing circuit 410 senses cardiac activity signals using a second sensing channel that includes the first LV electrode as a sensing cathode. Thus, each of the sensing channels is configured to share a cathode with one of the pacing channels. This is shown in FIG. 11 at 1105.

At 1110, the capture detection sub-circuit 420 initiates a change in pulse energy level provided to the first and second pacing channels. In the example of FIG. 11, the pulse energy level is stepped up from a pulse energy level at which cardiac capture (by either pacing channel) is not evident in sensed cardiac activity signals. Alternatively, the capture detection sub-circuit 420 may initiate the capture test with a pulse energy level that ensures cardiac capture by both pacing channels and the pulse energy level is then stepped down. Cardiac activity signals are sensed using both SCS channels after the pulse energy level is changed.

At 1115, the capture detection sub-circuit 420 uses the sensed signals to detect a change in the mode of cardiac capture Assuming a step up test, the capture detection sub-circuit 420 uses the sensed signals to detect a change from absence of capture to capture by one or both of the pacing channels. If the pulse energy was stepped down, the capture detection sub-circuit 420 check for a change from capture to loss of capture.

At 1120, the capture detection sub-circuit 420 checks whether a cardiac activity signal sensed using the first SCS channel displays a capture morphology associated with a shared cathode sensing. If the sensed signal does display a capture morphology, the pulse energy level associated with the detected change is recorded. At 1125, the cardiac capture pulse energy level threshold is determined for the first pacing channel using the recorded pulse energy level. In some examples, a safety margin is added to the recorded pulse energy level to determine the cardiac capture pulse energy level threshold. If the sensed signal does not display a capture morphology, the method returns to 1110 to continue stepping the pulse energy level.

At 1130, the capture detection sub-circuit 420 checks whether a cardiac activity signal sensed using the second SCS channel displays a capture morphology associated with a shared cathode sensing. If the sensed signal does display a capture morphology, the pulse energy level associated with the detected change is recorded.

At 1135, the cardiac capture pulse energy level threshold is determined for the first pacing channel. If the sensed signal does not display a morphology associated with cardiac capture, the method returns to 1110 to continue stepping the pulse energy level. The energy level is stepped until cardiac capture by both pacing channels is detected.

The methods in the examples of FIGS. 6 and 11 can be expanded from dual-site pacing to include more than two pacing sites. For instance, after the method is completed for two heart chamber electrodes e.g., LV electrodes 265 and 260 in FIG. 2A), the capture test can be run using another pair of electrodes (e.g., LV electrodes 264 and 262 in FIG. 2A) as pacing channels. Sensing channels can be configured to share the sensing cathode with a pacing channel cathode. For tri-site pacing, two pairs of electrodes can be selected with one electrode common to the pairs. The method can be performed for the first pair and then the second.

For more than quad-site pacing, pairs of the electrodes can he selected for pacing channels. A pair of pacing channels is selected for determining pace pulse energy thresholds. One or more cardiac signal sensing channel is selected that includes an LV electrode from the selected pair of pacing channels. Cardiac capture pulse energy level thresholds are determined for the selected pacing channels, pairs of the pacing channels continue to be selected and cardiac capture pulse energy level thresholds can be determined for the selected pairs until a cardiac capture pulse energy level threshold is determined for each of the pacing channels.

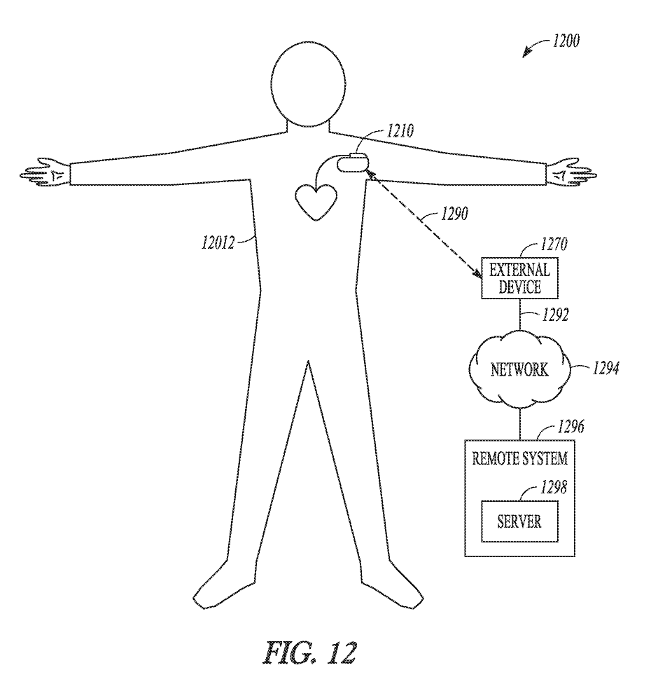

FIG. 12 is an illustration of portions of another system 1200 that uses an IMD 1210 to provide a therapy to a patient 1202. The system 1200 typically includes an external device 1270 that communicates signals 1290 wirelessly with a remote system 1296 via a network 1294. The network 1294 can be a communication network such as a phone network or a computer network (e.g., the internet). In some examples, the external device 1270 includes a repeater and communicated via the network using a link 1292. that may be wired or wireless. In some examples, the remote system 1296 provides patient management functions and may include one or more servers 1298 to perform the functions.

Returning to FIG. 4, the device 400 can include a communication circuit 435 to communicate information with a separate device, such as the external device 1270 of FIG. 12. When the cardiac capture pulse energy level thresholds are determined for the pacing channels by the capture detection sub-circuit 420, the control circuit 415 communicates indications of the cardiac capture pulse energy level thresholds to the separate device. In some variations, the indications may be stored in a memory of the device 400 and later uploaded by the separate device. In some variations, the indications could be communicated to the separate device in real time as the tests are performed. When the indications are communicated to the separate device they may be displayed to a user.

In some examples, the device 400 provides pacing therapy to the subject using the stimulus circuit 405 and the device updates the pacing thresholds for subsequent pacing therapy with the determined cardiac capture pulse energy level thresholds. some examples, the pacing thresholds are updated after a confirmation from the separate device is received. Because the pacing threshold values are optimized by the capture test, the updated pacing parameters provide effective therapy to the subject while optimizing battery life of the device.

Assistance by the CRM device in determining pacing thresholds can be especially beneficial if the clinician would like information concerning a large number or all of the possible pacing channels than can be configured by the device using deployed electrodes. The optimum stimulation threshold may vary over time for a patient due to maturation of myocardial tissue around an implanted electrode, drug therapy prescribed to the patient, an episode of myocardial infarction, and defibrillation of the myocardial tissue. Therefore, the tests may be run more than once by a device while the device is implanted.

The devices and methods described herein allow for multi-site stimulation to be delivered to the subject with the minimum stimulation energy required for effective therapy. The device may provide additional information related to the multi-site pacing to assist the clinician or caregiver in managing the device-based therapy.

### ADDITIONAL DESCRIPTION AND EXAMPLES

Example 1 includes subject matter (such as an apparatus) comprising: a stimulus circuit configured to provide electrical pulse energy to at least a first pacing channel that includes a first left ventricular (LV) electrode as a cathode and a second pacing channel that includes a second LV electrode as a cathode; a cardiac signal sensing circuit configured to sense cardiac activity signals using at least a first sensing channel that includes one of the first LV electrode or the second LV electrode; and a control circuit electrically coupled to the cardiac signal sensing circuit and the stimulus circuit, wherein the control circuit includes a capture detection sub-circuit configured to: initiate delivery of electrical pulse energy to both the first pacing channel and the second pacing channel; sense cardiac depolarization of a ventricle using the first sensing channel; determine a first cardiac capture pulse energy level threshold for the first pacing channel, and a second cardiac capture pulse energy level threshold for the second pacing channel; and provide indications of the first and second cardiac capture pulse energy level thresholds to a user or process

In Example 2, the subject matter of Example 1 optionally includes a capture detection sub-circuit configured to initiate a change in pulse energy level of the delivery of electrical pulse energy; and a control circuit that optionally includes a signal processing sub-circuit configured to identify a change in morphology in a cardiac activity signal sensed in association with a change from a first level of electrical pulse energy to a second level of electrical pulse energy. The capture detection sub-circuit is optionally further configured to distinguish, using the identified change in morphology, between cardiac capture by one of the first and second pacing channels from cardiac capture by both pacing channels.

In Example 3, the subject matter of Example 2 optionally includes a capture detection sub-circuit configured to distinguish, using the identified change in morphology, between cardiac capture by one or both of the first and second pacing channels from loss of capture by both pacing channels.

In Example 4, the subject matter of one or both of Examples 2 and 3 optionally include a capture detection sub-circuit configured to identify a change from capture by a pacing channel to loss of capture by the pacing channel when a cardiac activity signal sensed using a sensing channel that shares an electrode with the pacing channel indicates a change from a signal morphology indicating capture sensed using a cathode shared with a pacing channel to a signal morphology indicating absence of cardiac capture.

In Example 5, the subject matter of one any combination of Examples 2-4 optionally includes a capture detection sub-circuit configured to identify a change from capture by both of the first and second pacing channels to capture by one pacing channel when a cardiac activity signal sensed using a sensing channel that shares an electrode with the pacing channel indicates a change from a signal morphology indicating capture sensed using a cathode shared with a pacing channel to a signal morphology indicating capture sensed using a cathode independent from a pacing channel.

In Example 6, the subject matter of one or any combination of Examples 2-5 optionally includes a control circuit that includes a signal processing sub-circuit configured to identify a change in morphology in a sensed cardiac activity signal that indicates shared cathode sensing by a signal sensing channel and occurs in a time relation to the change in pulse energy level; and wherein the capture detection sub-circuit is configured to identify a change from absence of capture by a pacing channel to capture by the pacing channel when the change in morphology is identified.

In Example 7, the subject matter of one or any combination of Examples 1-6 optionally includes a first sensing channel that includes the first LV electrode as a sensing cathode, a cardiac signal sensing circuit configured to sense cardiac activity signals using a second sensing channel that includes the second LV electrode as a sensing cathode, and a capture detection sub-circuit is configured to: initiate changes in pulse energy level of pulse energy provided to the first and second pacing channels; monitor cardiac activity signals using only the first sensing channel; detect loss of capture by the first pacing channel using the first sensing channel; determine a first cardiac capture pulse energy level threshold for the first pacing channel using a pulse energy level associated with the loss of capture by the first pacing channel; change to monitoring cardiac activity signals using only the second sensing channel after the loss of capture by the first pacing channel is detected; detect loss of capture by the second pacing channel using the second sensing channel; and determine a second cardiac capture pulse energy level threshold for the second pacing channel using a pulse energy level associated with the loss of capture by the second pacing channel.

In Example 8, the subject matter of one or any combination of Examples 1-6 optionally includes a first sensing channel that includes the first LV electrode as a sensing cathode and is configured to sense a first cardiac activity signal; a cardiac signal sensing circuit configured to sense a second cardiac activity signal using a second sensing channel that includes the second LV electrode as a sensing cathode, and a capture detection sub-circuit configured to: initiate changes in pulse energy level of pulse energy provided to the first and second pacing channels; detect a change between capture and loss of capture by the first pacing channel using the first cardiac activity channel and determine the first cardiac capture pulse energy level threshold for the first pacing channel using a pulse energy level associated with the detected change; and detect a change between capture and loss of capture by the second pacing channel using the second cardiac activity channel and determine the second cardiac capture pulse energy level threshold for the second pacing channel using a pulse energy level associated with the detected change.

Example 9, the subject matter of one or any combination of Examples 1-6 optionally includes an implantable housing, wherein the first sensing channel includes the first LV electrode as the sensing cathode and an electrode formed on the implantable housing as the sensing anode.

In Example 10, the subject matter of one or any combination of Examples 1-6 optionally includes a cardiac signal sensing circuit configured to be electrically coupled to a right ventricular (RV) electrode, and wherein the first sensing channel includes the first LV electrode as the sensing cathode and the RV electrode as the sensing anode.

In Example 1, the subject matter of one or any combination of Examples 1-10 optionally includes a stimulus circuit configured to provide the electrical pulse energy to more than two pacing channels, wherein each pacing channel includes a different LV electrode, and a capture detection sub-circuit configured to: select a pair of the pacing channels; select a cardiac signal sensing channel that includes an LV electrode from the selected pair of pacing channels; determine cardiac capture pulse energy level thresholds for the selected pacing channels; and continue to select pairs of the pacing channels and determine cardiac capture pulse energy level thresholds for the selected pairs until a cardiac capture pulse energy level threshold is determined for each of the pacing channels.

Example 12 includes subject matter (such as a method of operating an ambulatory medical device, a means for performing acts, or a machine-readable medium including instructions that, when performed by the machine, cause the machine to perform acts), or can optionally be combined with the subject matter of one or any combination of Examples 1-11 to include such subject matter, comprising delivering electrical pulse energy to at least a first pacing channel of the MD that includes a first left ventricular (LV) electrode as a cathode and a second pacing channel of the IMD that includes a second LV electrode as a cathode; sensing cardiac depolarization of a ventricle using at least a first sensing channel that includes one of the first LV electrode or the second LV electrode; determining a first cardiac capture pulse energy level threshold for the first pacing channel, and a second cardiac capture pulse energy level threshold for the second pacing channel; and providing indications of the first and second cardiac capture pulse energy level thresholds to a user or process.

In Example 13, the subject matter of Example 12 optionally includes changing a pulse energy level of the delivered of electrical pulse energy; identifying a change in morphology in a cardiac activity signal sensed in association with the change in pulse energy level; and distinguishing, using the identified change in morphology, cardiac capture by one of the first and second pacing channels from cardiac capture by both pacing channels.

In Example 14, the subject matter of Example 13 optionally includes distinguishing, using the identified change in morphology, cardiac capture by one or both of the first and second pacing channels from loss of capture by both pacing channels.

In Example 15, the subject matter of one or both of Examples 13 and 14 optionally includes identifying a change from capture by a pacing channel to loss of capture by the pacing channel when a cardiac activity signal sensed using a sensing channel that shares an electrode with the pacing channel indicates a change from a signal morphology indicating capture sensed using a cathode shared with a pacing channel to a signal morphology indicating absence of cardiac capture.

In Example 16, the subject matter of one or any combination of Examples 13-15 optionally includes identifying a change from capture by both of the first and second pacing channels to capture by one pacing channel when a cardiac activity signal sensed using a sensing channel that shares an electrode with the one pacing channel indicates a change from a signal morphology indicating capture sensed using a cathode shared with a pacing channel to a signal morphology indicating capture sensed using a cathode independent from a pacing channel.

Example 17, the subject matter of one or any combination of Examples 12-16 optionally includes changing a pulse energy level of the delivered of electrical pulse energy; sensing cardiac depolarization of the ventricle using a first sensing channel that includes the first LV electrode as a sensing cathode and sensing cardiac depolarization of the ventricle using a second sensing channel that includes the second LV electrode as the sensing cathode, monitoring cardiac activity signals using only the first sensing channel; detecting loss of capture by the first pacing channel using the first sensing channel; determining a first cardiac capture pulse energy level threshold for the first pacing channel using a pulse energy level associated with the loss of capture by the first pacing channel; monitoring cardiac activity signals using only the second sensing channel after the loss of capture by the first pacing channel is detected; detecting loss of capture by the second pacing channel using the second sensing channel; and determining a second cardiac capture pulse energy level threshold for the second pacing channel using a pulse energy level associated with the loss of capture by the second pacing channel

In Example 18, the subject matter of one or any combination of Examples 12-17 optionally includes changing a pulse energy level of the delivered of electrical pulse energy; sensing cardiac depolarization of the ventricle using a first sensing channel that includes the first LV electrode as a sensing cathode and sensing cardiac depolarization of the ventricle using a second sensing channel that includes the second LV electrode as the sensing cathode; monitoring cardiac activity signals using the first sensing channel and the second sensing channel; detecting a change between capture and loss of capture by the first pacing channel using the first cardiac activity channel and determining the first cardiac capture pulse energy le threshold for the first pacing channel using a pulse energy level associated with the detected change; and detecting a change between capture and loss of capture by the second pacing channel using the second cardiac activity channel and determining the second cardiac capture pulse energy level threshold for the second pacing channel using a pulse energy level associated with the detected change.

Example 19 includes subject matter (such as a system), or can optionally be combined with the subject matter of one or any combination of Examples 1-18 to include such subject matter, comprising a plurality of implantable electrodes including a plurality of electrodes implantable in a left ventricle; and a first implantable device electrically coupled to the plurality of implantable electrodes. The first implantable optionally includes: a communication circuit configured to communicate information with a separate device; a stimulus circuit configured to provide electrical pulse energy to at least a first pacing channel that includes a first left ventricular (LV) electrode as a cathode and a second pacing channel that includes a second LV electrode as a cathode; a cardiac signal sensing circuit configured to sense cardiac activity signals using at least a first sensing channel that includes one of the first LV electrode or the second LV electrode; a control circuit electrically coupled to the cardiac signal sensing circuit and the stimulus circuit. The control circuit includes a capture detection sub-circuit configured to: initiate delivery of electrical pulse energy to both the first pacing channel and the second pacing channel; sense cardiac depolarization of a ventricle using the first sensing channel; and determine a first cardiac capture pulse energy level threshold for the first pacing channel, and a second cardiac capture pulse energy level threshold for the second pacing channel, wherein the control circuit is configured to communicate indications of the first and second cardiac capture pulse energy level thresholds to the separate device.

In Example 20, the subject matter of Example 19 optionally includes an implantable lead that includes the plurality of implantable electrodes

Example 21 can include, or can optionally be combined with any portion or combination of any portions of any one or more of Examples 1-20 to include, subject matter that can include means for performing any one or more of the functions of Examples 1-20, or a machine-readable medium including instructions that when performed by a machine, cause the machine to perform any one or more of the functions of Examples 1-20.

These non-limiting examples can be combined in any permutation or combination.

The above detailed description includes references to the accompanying drawings, which form a part of the detailed description. The drawings show, by way of illustration, specific embodiments in which the invention can be practiced.

These embodiments are also referred to herein as “examples.” All publications, patents, and patent documents referred to in this document are incorporated by reference herein in their entirety, as though individually incorporated by reference. in the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus for electrical coupling to a plurality of implantable electrodes, the apparatus comprising:
a stimulus circuit configured to provide electrical pulse energy to at least a first pacing channel that includes a first left ventricular (LV) electrode as a cathode and a second pacing channel that includes a second LV electrode as a cathode;
a cardiac signal sensing circuit configured to sense cardiac activity signals using at least a first sensing channel that includes one of the first LV electrode or the second LV electrode;
a control circuit electrically coupled to the cardiac signal sensing circuit and the stimulus circuit, wherein the control circuit includes a capture detection sub-circuit configured to: initiate delivery of electrical pulse energy to both the first pacing channel and the second pacing channel; sense cardiac depolarization of a ventricle using the first sensing channel; determine a first cardiac capture pulse energy level threshold for the first pacing channel, and a second cardiac capture pulse energy level threshold for the second pacing channel; and provide indications of the first and second cardiac capture pulse energy level thresholds to a user or process.

2. The apparatus of claim 1,
wherein the capture detection sub-circuit is configured to initiate a change in pulse energy level of the delivery of electrical pulse energy;
wherein the control circuit includes a signal processing sub-circuit configured to identify a change in morphology in a cardiac activity signal sensed in association with a change from a first level of electrical pulse energy to a second level of electrical pulse energy; and
wherein the capture detection sub-circuit is further configured to distinguish, using the identified change in morphology, between cardiac capture by one of the first and second pacing channels from cardiac capture by both pacing channels.

3. The apparatus of claim 2, wherein the capture detection sub-circuit is configured to distinguish, using the identified change in morphology, between cardiac capture by one or both of the first and second pacing channels from loss of capture by both pacing channels.

4. The apparatus of claim 2, wherein the capture detection sub-circuit is configured to identify a change from capture by a pacing channel to loss of capture by the pacing channel when a cardiac activity signal sensed using a sensing channel that shares an electrode with the pacing channel indicates a change from a signal morphology indicating capture sensed using a cathode shared with a pacing channel to a signal morphology indicating absence of cardiac capture.

5. The apparatus of claim 2, wherein the capture detection sub-circuit is configured to identify a change from capture by both of the first and second pacing channels to capture by one pacing channel when a cardiac activity signal sensed using a sensing channel that shares an electrode with the pacing channel indicates a change from a signal morphology indicating capture sensed using a cathode shared with a pacing channel to a signal morphology indicating capture sensed using a cathode independent from a pacing channel.

6. The apparatus of claim 2, wherein the control circuit includes a signal processing sub-circuit configured to identify a change in morphology in a sensed cardiac activity signal that indicates shared cathode sensing by a signal sensing channel and occurs in a time relation to the change in pulse energy level; and wherein the capture detection sub-circuit is configured to identify a change from absence of capture by a pacing channel to capture by the pacing channel when the change in morphology is identified.

7. The apparatus of claim 1,
wherein the first sensing channel includes e first LV electrode as a sensing cathode,
wherein the cardiac signal sensing circuit is further configured to sense cardiac activity signals using a second sensing channel that includes the second LV electrode as a sensing cathode,
wherein the capture detection sub-circuit is configured to:
initiate changes in pulse energy level of pulse energy provided to the first and second pacing channels;
monitor cardiac activity signals using only the first sensing channel;
detect loss of capture by the first pacing channel using the first sensing channel;
determine a first cardiac capture pulse energy level threshold for the first pacing channel using a pulse energy level associated with the loss of capture by the first pacing channel;
change to monitoring cardiac activity signals using only the second sensing channel after the loss of capture by the first pacing channel is detected;
detect loss of capture by the second pacing channel using the second sensing channel; and
determine a second cardiac capture pulse energy level threshold for the second pacing channel using a pulse energy level associated with the loss of capture by the second pacing channel.

8. The apparatus of claim 1,
wherein the first sensing channel includes the first LV electrode as a sensing cathode and is configured to sense a first cardiac activity signal;
wherein the cardiac signal sensing circuit is further configured to sense a second cardiac activity signal using a second sensing channel that includes the second LV electrode as a sensing cathode,
wherein the capture detection sub-circuit is configured to:
initiate changes in pulse energy level of pulse energy provided to the first and second pacing channels;
detect a change between capture and loss of capture by the first pacing channel using the first cardiac activity channel and determine the first cardiac capture pulse energy level threshold for the first pacing channel using a pulse energy level associated with the detected change; and
detect a change between capture and loss of capture by the second pacing channel using the second cardiac activity channel and determine the second cardiac capture pulse energy level threshold for the second pacing channel using a pulse energy level associated with the detected change.

9. The apparatus of claim 1, including an implantable housing, wherein the first sensing channel includes the first LV electrode as the sensing cathode and an electrode formed on the implantable housing as the sensing anode.

10. The apparatus of claim 1, wherein the cardiac signal sensing circuit is configured to be electrically coupled to a right ventricular (RV) electrode, and wherein the first sensing channel includes the first LV electrode as the sensing cathode and the RV electrode as the sensing anode.

11. The apparatus of claim 1, wherein the stimulus circuit is configured to provide the electrical pulse energy to more than two pacing channels, wherein each pacing channel includes a different LV electrode, and wherein the capture detection sub-circuit is configured to:
select a pair of the pacing channels;
select a cardiac signal sensing channel that includes an LV electrode from the selected pair of pacing channels;
determine cardiac capture pulse energy level thresholds for the selected pacing channels; and
continue to select pairs of the pacing channels and determine cardiac capture pulse energy level thresholds for the selected pairs until a cardiac capture pulse energy level threshold is determined for each of the pacing channels.

12. A method of controlling operation of an implantable medical device (IMD), the method comprising:
delivering electrical pulse energy to at least a first pacing channel of the MID that includes a first left ventricular (LV) electrode as a cathode and a second pacing channel of the IMD that includes a second LV electrode as a cathode;
sensing cardiac depolarization of a ventricle using at least a first sensing channel that includes one of the first LV electrode or the second LV electrode;
determining a first cardiac capture pulse energy level threshold for the first pacing channel, and a second cardiac capture pulse energy level threshold for the second pacing channel; and
providing indications of the first and second cardiac capture pulse energy level thresholds to a user or process.

13. The method of claim 12, including:
changing a pulse energy level of the delivered of electrical pulse energy;
identifying a change in morphology in a cardiac activity signal sensed in association with the change in pulse energy level; and
distinguishing, using the identified change in morphology, cardiac capture by one of the first and second pacing channels from cardiac capture by both pacing channels.

14. The method of claim 13, including distinguishing, using the identified change in morphology, cardiac capture by one or both of the first and second pacing channels from loss of capture by both pacing channels.

15. The method of claim 13, wherein identifying a change in morphology includes identifying a change from capture by a pacing channel to loss of capture by the pacing channel when a cardiac activity signal sensed using a sensing channel that shares an electrode with the pacing channel indicates a change from a signal morphology indicating capture sensed using a cathode shared with a pacing channel to a signal morphology indicating absence of cardiac capture.

16. The method of claim 13, wherein identifying a change in morphology includes identifying a change from capture by both of the first and second pacing channels to capture by one pacing channel when a cardiac activity signal sensed using a sensing channel that shares an electrode with the one pacing channel indicates a change from a signal morphology indicating capture sensed using a cathode shared with a pacing channel to a signal morphology indicating capture sensed using a cathode independent from a pacing channel.

17. The method of claim 12, including:
changing a pulse energy level of the delivered of electrical pulse energy;
sensing cardiac depolarization of the ventricle using a first sensing channel that includes the first LV electrode as a sensing cathode and sensing cardiac depolarization of the ventricle using a second sensing channel that includes the second LV electrode as the sensing cathode;
monitoring cardiac activity signals using only the first sensing channel;
detecting loss of capture by the first pacing channel using the first sensing channel;
determining a first cardiac capture pulse energy level threshold for the first pacing channel using a pulse energy level associated with the loss of capture by the first pacing channel;
monitoring cardiac activity signals using only the second sensing channel after the loss of capture by the first pacing channel is detected;
detecting loss of capture by the second pacing channel using the second sensing channel; and
determining a second cardiac capture pulse energy level threshold for the second pacing channel using a pulse energy level associated with the loss of capture by the second pacing channel.

18. The method of claim 12,
changing a pulse energy level of the delivered of electrical pulse energy;
sensing cardiac depolarization of the ventricle using a first sensing channel that includes the first LV electrode as a sensing cathode and sensing cardiac depolarization of the ventricle using a second sensing channel that includes the second LV electrode as the sensing cathode;
monitoring cardiac activity signals using the first sensing channel and the second sensing channel;
detecting a change between capture and loss of capture by the first pacing channel using the first cardiac activity channel and determining the first cardiac capture pulse energy level threshold for the first pacing channel using a pulse energy level associated with the detected change; and
detecting a change between capture and loss of capture by the second pacing channel using the second cardiac activity channel and determining the second cardiac capture pulse energy level threshold for the second pacing channel using a pulse energy level associated with the detected change.

19. A system comprising:
a plurality of implantable electrodes including a plurality of electrodes implantable in a left ventricle; and
a first implantable device electrically coupled to the plurality of implantable electrodes, wherein the first implantable includes:
a communication circuit configured to communicate information with a separate device;
a stimulus circuit configured to provide electrical pulse energy to at least a first pacing channel that includes a first left ventricular (LV) electrode as a cathode and a second pacing channel that includes a second LV electrode as a cathode;
a cardiac signal sensing circuit configured to sense cardiac activity signals using at least a first sensing channel that includes one of the first LV electrode or the second LV electrode;
a control circuit electrically coupled to the cardiac signal sensing circuit and the stimulus circuit, wherein the control circuit includes a capture detection sub-circuit configured to:
initiate delivery of electrical pulse energy to both the first pacing channel and the second pacing channel;
sense cardiac depolarization of a ventricle g the first sensing channel; and
determine a first cardiac capture pulse energy level threshold for the first pacing channel, and a second cardiac capture pulse energy level threshold for the second pacing channel, wherein the control circuit is configured to communicate indications of the first and second cardiac capture pulse energy level thresholds to the separate device.

20. The system of claim 19, including an implantable lead, wherein the implantable lead includes the plurality of implantable electrodes.
